(19) United States
(12) Patent Application Publication
(10) Pub. No.: US 2017/0357930 A1
(43) Pub. Date: Dec. 14, 2017

(54) WORK PLAN SUPPORT INFORMATION PROVISION METHOD, WORK PLAN SUPPORT INFORMATION PROVISION APPARATUS, AND COMPUTER-READABLE RECORDING MEDIUM

(71) Applicants: FUJITSU LIMITED, Kawasaki-shi (JP); TRANSTRON INC., Yokohama-shi (JP)

(72) Inventors: Hiroyuki Tani, Katsushika (JP); Sei Masuda, Kagoshima (JP); Mitsuru Tanaka, Yokohama (JP); Masayoshi Hoshiya, Yokohama (JP); Yuuki Murase, Yokohama (JP); Masatsugu Isogai, Yokohama (JP)

(73) Assignees: FUJITSU LIMITED, Kawasaki-shi (JP); TRANSTRON INC., Yokohama-shi (JP)

(21) Appl. No.: 15/617,179

(22) Filed: Jun. 8, 2017

(30) Foreign Application Priority Data
Jun. 9, 2016 (JP) 2016-115041

(51) Int. Cl. G06Q 10/06

(52) U.S. Cl. CPC G06Q 10/063114; G06Q 10/063116

(57) ABSTRACT

A work plan support information provision method includes: calculating fatigue degrees of an individual for a plurality of types of operations in a future work time zone including the types of operations based on a past work result of the individual and attribute information of the individual by referring to a storage unit storing therein the past work result and the attribute information, by a processor; and outputting the calculated fatigue degrees so as to correspond to the respective types of operations included in the future work time zone, by the processor.

### CROSS-REFERENCE TO RELATED APPLICATION

This application is based upon and claims the benefit of priority of the prior Japanese Patent Application No. 2016-115041, filed on Jun. 9, 2016, the entire contents of which are incorporated herein by reference.

### Field

The embodiments discussed herein are related to a work plan support information provision method, a computer-readable recording medium, and a work plan support information provision apparatus.

### Background

Conventionally, techniques of previously specifying a break time and a break place to a driver have been proposed.

Japanese Laid-open Patent Publication No. 2015-125611 is an example of the related techniques.

A state in which a driver is prompted to take a break with the related technique possibly leads to a dangerous state for driving due to fatigue before the break specified time when the driver is specified to take a break because a fatigue state of the driver is difficult to grasp. Eventually, occurrence of an accident can be unpreventable in some cases. That is to say, the related technique does not predict a driver's physical condition. For this reason, a good decision is not made on whether work that a person in charge of allocation plans to allocate to a certain driver is adequate, for example, at the stage of allocation of the work to the driver.

### SUMMARY

According to an aspect of the embodiments, a work plan support information provision method includes: calculating fatigue degrees of an individual for a plurality of types of operations in a future work time zone including the types of operations based on a past work result of the individual and attribute information of the individual by referring to a storage unit storing therein the past work result and the attribute information, by a processor; and outputting the calculated fatigue degrees so as to correspond to the respective types of operations included in the future work time zone, by the processor.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for explaining an example of a system configuration;

FIG. 2 is a diagram illustrating an example of an operation recording meter;

FIG. 3 is a diagram illustrating an example of a data structure of operation information;

FIG. 4 is a diagram illustrating an example of a data structure of state information;

FIG. 5 is a diagram illustrating an example of a work plan support information provision apparatus;

FIG. 6 is a diagram illustrating an example of a data structure of work result information;

FIG. 7 is a diagram illustrating an example of a data structure of user attribute information;

FIG. 8 is a diagram illustrating an example of a data structure of operation plan information;

FIG. 9 is a diagram illustrating an example of an operation plan registration screen;

FIG. 10 is a diagram illustrating an example of a user attribute registration screen;

FIG. 11A is a diagram illustrating an example of calculation of a driving time;

FIG. 11B is a diagram illustrating an example of calculation of a break time;

FIG. 11C is a diagram illustrating an example of calculation of a rest time;

FIG. 11D is a diagram illustrating an example of calculation of a standby time;

FIG. 11E is a diagram illustrating an example of calculation of a loading time;

FIG. 11F is a diagram illustrating an example of calculation of an unloading time;

FIG. 11G is a diagram illustrating an example of calculation of a work interval time;

FIG. 11H is a diagram illustrating an example of calculation of an on-duty time;

FIG. 12A is a diagram illustrating an example of calculation of a fatigue degree at the time of return;

FIG. 12B is a diagram illustrating an example of calculation of the fatigue degree at the time of leaving;

FIG. 12C is a diagram illustrating another example of calculation of the fatigue degree at the time of return;

FIG. 13 is a diagram illustrating an example of a fatigue degree presentation screen;

FIG. 14 is a flowchart illustrating an example of procedures of work plan support information provision processing;

FIG. 15A is a diagram illustrating an example of calculation of the fatigue degree when drowsiness has been detected;

FIG. 15B is a diagram illustrating an example of calculation of the fatigue degree in real time;

FIG. 15C is a diagram illustrating an example of modification of an operation plan in accordance with the fatigue degree;

FIG. 15D is a diagram illustrating another example of modification of the operation plan in accordance with the fatigue degree; and

FIG. 16 is a diagram illustrating an example of the configuration of a computer executing a work plan support information provision program.

### DESCRIPTION OF EMBODIMENTS

Preferred embodiments will be explained with reference to accompanying drawings. It is to be noted that the embodiments do not limit a disclosed technique. The embodiments that will be described below may be appropriately combined within a consistent range.

### [a] First Embodiment

System Configuration

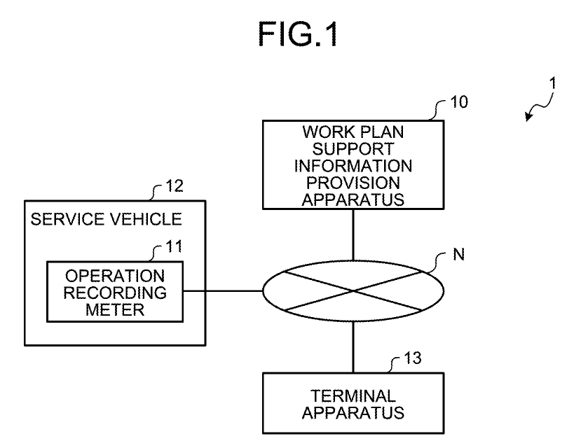

In, for example, the transportation industry, an operation recording meter for monitoring an operation state, such as a digital tachograph, is mounted on a service vehicle and operation management is performed based on pieces of information collected from the operation recording meter. An example of a system that performs operation management in a first embodiment will be described. FIG. 1 is a diagram for explaining an example of a system configuration. As illustrated in FIG. 1, a system 1 includes a work plan support information provision apparatus 10, an operation recording meter 11, and a terminal apparatus 13. The work plan support information provision apparatus 10, the operation recording meter 11, and the terminal apparatus 13 are connected to a network N in a communicable manner. As a mode of the network N, a desired type of communication network such as mobile communication using cellular phones and the like, the Internet, the local area network (LAN), and the virtual private network (VPN) can be employed regardless of wired or wireless communication.

The operation recording meter 11 is an apparatus that is mounted, for example, in the vicinity of a driver's seat of a vehicle, and monitors and records operation of the vehicle on which it is mounted. The operation recording meter 11 is mounted on a service vehicle 12 such as a track and a bus. It is to be noted that although the service vehicle 12 on which the operation recording meter 11 is mounted is one in the example of FIG. 1, the number of service vehicles 12 is not limited thereto and the desired number of service vehicles 12 can be provided.

The terminal apparatus 13 is, for example, a terminal apparatus such as a personal computer that is arranged in an office of a transportation company or the like. The terminal apparatus 13 is used when, for example, a person in charge of the operation management in the office accesses the work plan support information provision apparatus 10 to perform the operation management of the service vehicle 12.

The work plan support information provision apparatus 10 is an apparatus that performs the operation management of the service vehicle 12. The work plan support information provision apparatus 10 provides information supporting creation of a work plan such as an operation plan of the service vehicle 12. The work plan support information provision apparatus 10 is, for example, a computer such as a personal computer and a server computer. The work plan support information provision apparatus 10 may be implemented as one computer or a plurality of computers. For example, an apparatus that manages the operation of the service vehicle 12 and an apparatus that provides the information supporting creation of the work plan can be implemented as different computers. It is to be noted that in the embodiment, the work plan support information provision apparatus 10 is implemented as one computer, as an example.

The work plan support information provision apparatus 10 performs the operation management. The work plan support information provision apparatus 10 collects various pieces of information on a driver that the operation recording meter 11 has acquired through the network N. The work plan support information provision apparatus 10 performs the operation management of the service vehicle 12 based on the collected pieces of information. In the example of FIG. 1, the work plan support information provision apparatus 10 collects the various pieces of information from the operation recording meter 11 through the network N. The work plan support information provision apparatus 10 is however not limited to collect them in this manner. The work plan support information provision apparatus 10 may collect the various pieces of information that the operation recording meter 11 has acquired through, for example, a recording medium such as a flash memory. Alternatively, the work plan support information provision apparatus 10 may collect the various pieces of information that the operation recording meter 11 has acquired through, for example, wired communication or wireless communication with the operation recording meter 11.

Configuration of Operation Recording Meter

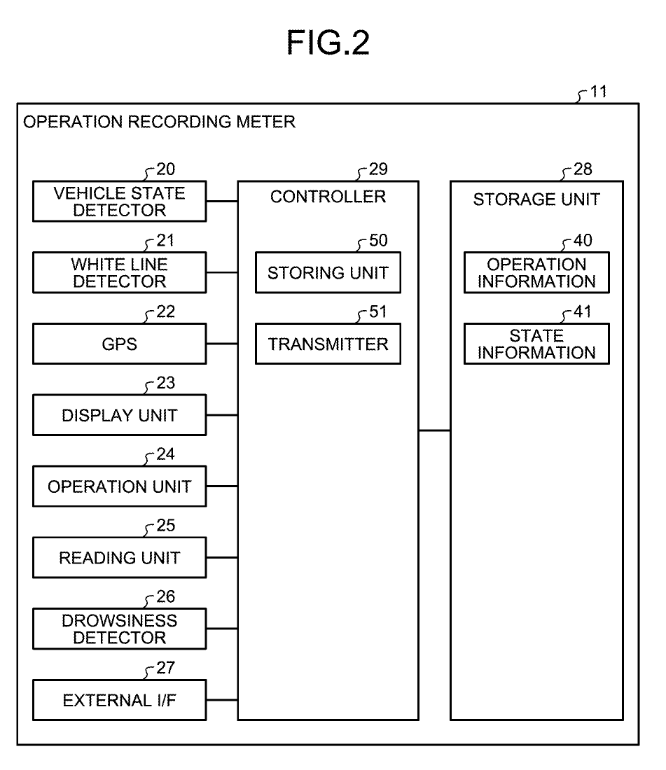

Next, the configurations of the respective apparatuses will be described. First, the configuration of the operation recording meter 11 will be described. FIG. 2 is a diagram illustrating an example of the operation recording meter. The operation recording meter 11 illustrated in FIG. 2 includes a vehicle state detector 20, a white line detector 21, and a global positioning system (GPS) 22. Furthermore, the operation recording meter 11 includes a display unit 23, an operation unit 24, a reading unit 25, a drowsiness detector 26, an external interface (I/F) 27, a storage unit 28, and a controller 29.

The vehicle state detector 20 is a detector that detects various states related to the service vehicle 12. The vehicle state detector 20 detects, for example, a traveling speed and a traveling distance of the vehicle based on signals from a speed sensor provided in the vehicle. The traveling distance is measured as a cumulative value. The white line detector 21 is a detector that detects deviation of the vehicle from a white line. The white line detector 21, for example, detects white lines indicating lanes of roads by image analysis of captured images provided by a camera directed to the front of the vehicle to detect deviation of the vehicle from a white line. The GPS 22 measures a current position of the vehicle based on signals from GPS satellite.

The display unit 23 is a device that performs various displays. The display unit 23 is, for example, a display device such as a liquid crystal display installed at a position enabling the driver on the driver's seat of the service vehicle 12 to view it. The display unit 23 displays various messages such as a warning message. Furthermore, the display unit 23 displays various operation screens in accordance with operations through the operation unit 24. The display unit 23 displays, for example, an operation screen for specifying an operation state in accordance with an operation through the operation unit 24.

The operation unit 24 is an input device such as buttons and a touch panel that receives various operation inputs. For example, a status switch for specifying the operation state is provided in the operation unit 24. The operation unit 24 enables specification of the operation states such as non-specification, driving, loading, unloading, break, rest, and return. The non-specification indicates the operation state, for example, when the driver does not drive the vehicle but temporarily stands by in the vehicle so as to drive it. The break indicates the operation state, for example, when the driver takes a short break for a meal or the like. The rest indicates the operation state, for example, when the driver takes a long break for a nap or the like. It is to be noted that usage manners of the non-specification state, the break, and the rest are not limited thereto. Furthermore, the operation states may include standby. The operation states may include various other states. For example, leaving may be able to be specified as the operation state. The operation unit 24 may be configured such that switches are provided for the respective operation states to enable specification of the respective operation states. Alternatively, the operation unit 24 may enable specification of the respective operation states by combinations of a plurality of buttons. The operation unit 24 may be configured, for example, by switch buttons for switching the operation state, such as up and down keys and a cross key, and a determination button for determining the operation state. When any one operation state is selected, the operation recording meter 11 in the embodiment treats the selected operation state as lasting in a period until a subsequent operation state is selected. For example, when driving is selected, the operation recording meter 11 treats the operation state of driving as lasting until another operation state is selected. Furthermore, the operation recording meter 11 in the embodiment treats, as leaving, the timing of first selection of the operation state of driving after return is selected. The operation recording meter 11 may be configured to enable specification of start and end of the respective operation states. For example, the operation state may be treated as being started based on detection that the driver presses a button indicating “leaving”.

The reading unit 25, for example, executes non-contact IC communication with a non-contact IC card storing therein user identification (ID) and reads the user ID stored in the non-contact IC card to acquire the user ID. For example, a driver's license can also be used as the non-contact IC card. Personal information such as a driver's license number stored in the driver's license may be used as the user ID. The reading unit 25, for example, executes non-contact IC communication with the driver's license and reads the personal information in the driver's license to acquire the read personal information as the user ID. The user ID may be input through the operation unit 24.

The drowsiness detector 26 is a detector that detects generation of drowsiness. The drowsiness detector 26 detects the drowsiness of the driver, for example, by analyzing fluctuations of driver's pulses measured by a contact-type pulse measurement unit of an earring type that the driver wears on an ear or a non-contact-type pulse measurement unit. The pulses may be detected by a method other than direct contact. The drowsiness detector 26 may detect the driver's pulses, for example, by emitting radio waves to the driver and detecting fluctuations of reflection conditions of the radio waves.

The external I/F 27 is, for example, an interface for transmitting and receiving various pieces of information to and from other devices. In the operation recording meter 11, the external I/F 27 is a wireless communication interface for making wireless communication with the network N. When the operation recording meter 11 transmits and receives various pieces of information to and from the work plan support information provision apparatus 10 through a recording medium, the external I/F 27 is a port for inputting and outputting pieces of data to the recording medium. When the operation recording meter 11 transmits and receives various pieces of information to and from the work plan support information provision apparatus 10 by wired communication or wireless communication, the external I/F 27 is a communication interface for making wired communication or wireless communication.

The storage unit 28 is a storage device such as a hard disk, a solid state drive (SSD), and an optical disk. The storage unit 28 may be a data rewritable semiconductor memory such as a random access memory (RAM), a flash memory, and a non volatile static random access memory (NVSRAM). The storage unit 28 stores therein an operating system (OS) and various computer programs that the controller 29 executes. The storage unit 28 stores therein various pieces of information. The storage unit 28 stores therein, for example, operation information 40 and state information 41.

The operation information 40 is data storing various pieces of information related to the operation of the vehicle. The operation information 40 stores various pieces of data detected by the vehicle state detector 20, the white line detector 21, and the GPS 22.

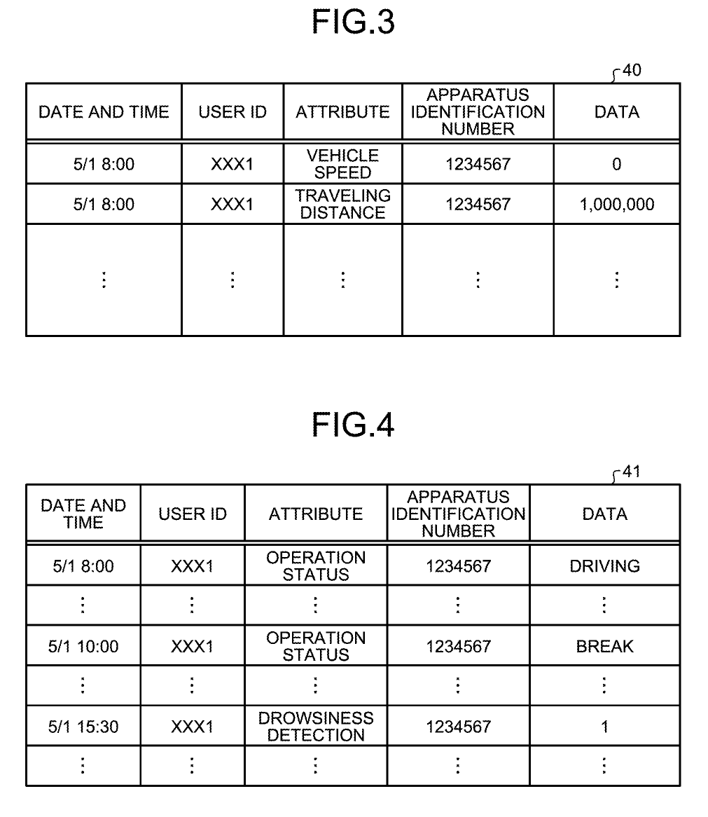

FIG. 3 is a diagram illustrating an example of a data structure of the operation information. As illustrated in FIG. 3, the operation information 40 has items of a date and time, user ID, an attribute, an apparatus identification number, and data. The item of the date and time is a region storing therein the date and time when the data has been detected. The item of the user ID is a region storing therein identification information of the driver that operates the vehicle. The item of the user ID is a region storing therein the user ID of the driver that the reading unit 25 has read. The item of the attribute is a region storing therein identification information indicating a type of the detected data. Although the data type is expressed by a name for making the data type easy to be known in the embodiment, for example, an attribute code is stored as the identification information indicating the data type. The item of the apparatus identification number is a region storing therein identification information for identifying the operation recording meter 11. A unique apparatus identification number is assigned to the operation recording meter 11 as the identification information for identifying each operation recording meter 11. The apparatus identification number assigned to the operation recording meter 11 is stored in the item of the apparatus identification number. The item of the data is an item in which the detected data is stored. The detected data is stored in the item of the data. When the attribute is, for example, a vehicle speed, a value of a speed per hour [km/h] is stored in the item of the data. When the attribute is a traveling distance, a cumulative value of the traveling distance [m] is stored in the item of the data. When the attribute is deviation from a white line, “1” is stored in the item of the data in the case in which the white line detector 21 detects the deviation from a white line. When the attribute is a position that the GPS 22 measures, positional information that the GPS 22 measures is stored in the item of the data.

The example of FIG. 3 indicates that a driver the user ID of which is “XXX1” drives the service vehicle 12 and the apparatus identification number of the operation recording meter 11 is “1234567”. The example of FIG. 3 further indicates that the vehicle speed has been detected at 8:00 on May 1 and the detected vehicle speed is 0 [km/h]. Moreover, the example of FIG. 3 indicates that the traveling distance has been detected at 8:00 on May 1 and the detected traveling distance is 1,000,000 [m].

The state information 41 is data storing various piece of information related to a state of the driver and the operation state. The state information 41 stores information indicating the operation state specified through the operation unit 24 and various pieces of data detected by the drowsiness detector 26.

FIG. 4 is a diagram illustrating an example of a data structure of the state information. The state information 41 has a data structure that is similar to that of the operation information 40. In the example of FIG. 4, an attribute indicating the operation state is defined as “operation status”. An attribute of detection of drowsiness by the drowsiness detector 26 is defined as “drowsiness detection”. Detected data is stored in the item of the data. When the attribute is, for example, the drowsiness detection, “1” is stored in the item of the data in the case in which the drowsiness detector 26 detects the drowsiness. When the attribute is the operation status, the operation state specified through the operation unit 24 is stored in the item of the data. Although the operation state is expressed by a name for making the operation state easy to be known in the embodiment, for example, an attribute code indicating the operation state is stored.

The example of FIG. 4 indicates that the driver the user ID of which is “XXX1” drives the service vehicle 12 and the apparatus identification number of the operation recording meter 11 is “1234567”. The example of FIG. 4 further indicates that “driving” has been specified as the operation state at 8:00 on May 1. The operation state of driving corresponds to leaving when it is a first selected operation state after the previous return. Moreover, the example of FIG. 4 indicates that “break” has been specified as the operation state at 10:00 on May 1. The example of FIG. 4 also indicates that the drowsiness detector 26 has detected the drowsiness at 15:30 on May 1.

The data structures of the operation information 40 and the state information 41 illustrated in FIG. 3 and FIG. 4, respectively, are examples and not limited thereto. The operation information 40 and the state information 41 may be famed by, for example, one file. Alternatively, the operation information 40 and the state information 41 may be formed by separate files for the respective attributes. The operation information 40 and the state information 41 may have the data structures in which the pieces of data in the respective items are sectioned by predetermined section characters in the predetermined order. The operation information 40 and the state information 41 may have data structures in which data attributes are indicated using tags or the like.

The controller 29 controls the overall operation recording meter 11. As the controller 29, an electronic circuit such as a central processing unit (CPU) and a micro processing unit (MPU) or an integrated circuit such as an application specific integrated circuit (ASIC) and a field programmable gate array (FPGA) can be employed. The controller 29 has an internal memory for storing therein computer programs defining various processing procedures and pieces of control data and executes various pieces of processing by using them. The controller 29 functions as various processors by causing the various computer programs to be executed. The controller 29 includes, for example, a storing unit 50 and a transmitter 51.

The storing unit 50 stores various pieces of data detected by the vehicle state detector 20 and the white line detector 21 in the operation information 40. Furthermore, the storing unit 50 stores the information indicating the operation state specified through the operation unit 24 and the various pieces of data detected by the drowsiness detector 26 in the state information 41.

The transmitter 51 transmits the operation information 40 and the state information 41 to the work plan support information provision apparatus 10 at a predetermined timing.

Configuration of Work Plan Support Information Provision Apparatus

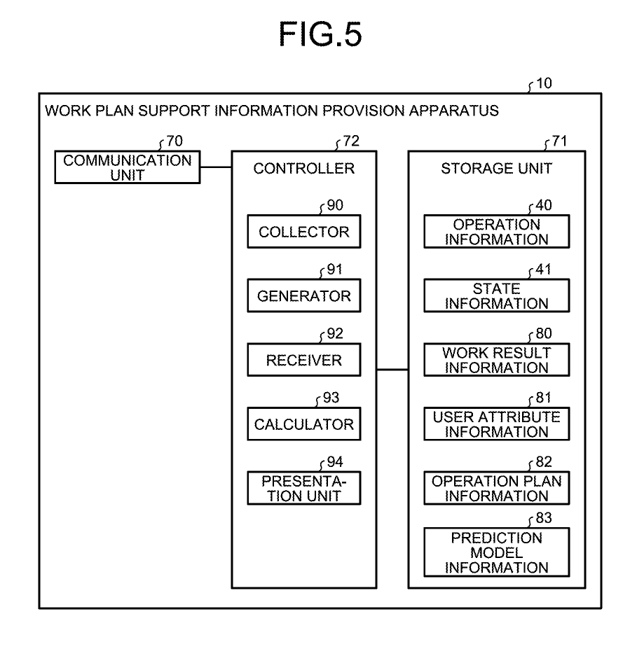

Next, the configuration of the work plan support information provision apparatus 10 will be described. FIG. 5 is a diagram illustrating an example of the work plan support information provision apparatus. The work plan support information provision apparatus 10 illustrated in FIG. 5 includes a communication unit 70, a storage unit 71, and a controller 72.

The communication unit 70 is, for example, a communication interface for making wireless communication or wired communication with the network N. The storage unit 71 is a storage device such as a hard disk, an SSD and an optical disk. The storage unit 71 may be a data rewritable semiconductor memory. The storage unit 71 stores therein an operating system (OS) and various computer programs that the controller 72 executes. The storage unit 71 stores therein various piece of information. The storage unit 71 stores therein, for example, the operation information 40, the state information 41, work result information 80, user attribute information 81, operation plan information 82, and prediction model information 83.

The operation information 40 and the state information 41 are collected from the operation recording meter 11 and stored.

The work result information 80 is data provided by processing the operation state from the state information 41 as a work result. For example, a generator 91, which will be described later, generates the work result information 80.

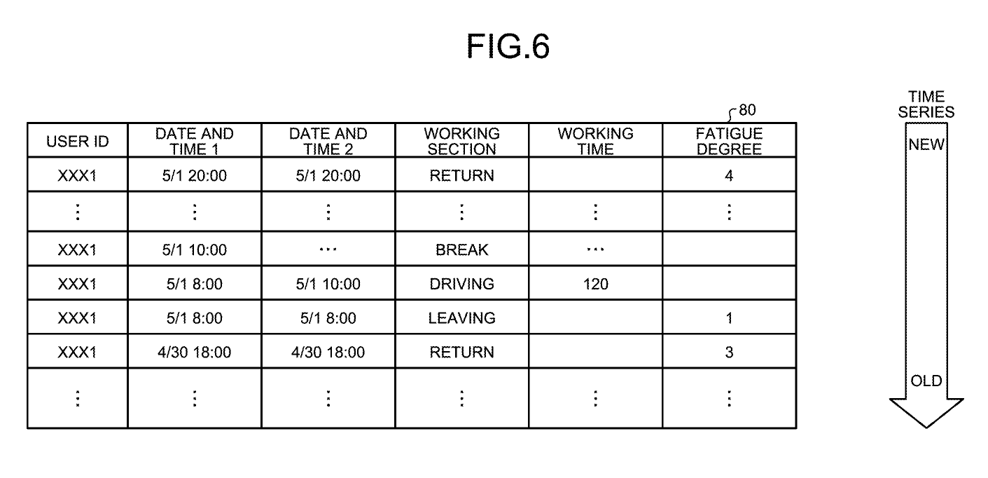

FIG. 6 is a diagram illustrating an example of a data structure of the work result information. The work result information 80 has items of user ID, a date and time 1, a date and time 2, a working section, a working time, and a fatigue degree. The item of the user ID is a region storing therein the user ID. The item of the date and time 1 is a region storing therein a start date and time of the operation state in the item of the working section. The item of the date and time 2 is a region storing therein an end date and time of the operation state in the item of the working section. The item of the working section is a region storing therein the operation state. The same date and time is stored in the item of the date and time 1 and the item of the date and time 2 at the timings when the operation state is leaving and return. The item of the working time is a region storing therein a time length (minute) from the date and time in the item of the date and time 1 to the date and time in the item of the date and time 2. The item of the fatigue degree is a region storing therein the fatigue degree indicating the degree of fatigue of the driver. In the embodiment, the degree of fatigue of the driver is expressed by the fatigue degrees of 1 to 5. As a value of the fatigue degree is higher, the degree of fatigue is higher. In the work plan support information provision apparatus 10 in the embodiment, an initial value of the fatigue degree is once set for each driver. The initial value of the fatigue degree may be a predetermined value (for example, 1).

The example of FIG. 6 indicates that the driver the user ID of which is “XXX1” has returned at 18:00 on April 30. The example of FIG. 6 indicates that the driver the user ID of which is “XXX1” has left at 8:00 on May 1 because the driver has selected the operation state of driving. For the driver the user ID of which is “XXX1”, the start date and time when the operation state is made into driving is 8:00 on May 1, the end date and time when the operation state of driving is ended is 10:00 on May 1, and the working time is 120 minutes.

The user attribute information 81 is data storing pieces of information on various attributes for each user. The user attribute information 81 stores therein, for example, pieces of information on various attributes that influence the fatigue for each user. In the embodiment, pieces of information such as a body mass index (BMI), age, and a sleeping time are stored as the various attributes that influence the fatigue. For example, even with the same work, the driver tends to fatigue as the age is higher. Even with the same work, the driver tends to fatigue as the BMI is farther from a standard weight range. The driver is difficult to recover from the fatigue as the sleeping time is shorter than a predetermined standard time (for example, eight hours). The attributes that influence the fatigue are not limited thereto and any attribute may be employed as long as it influences the fatigue.

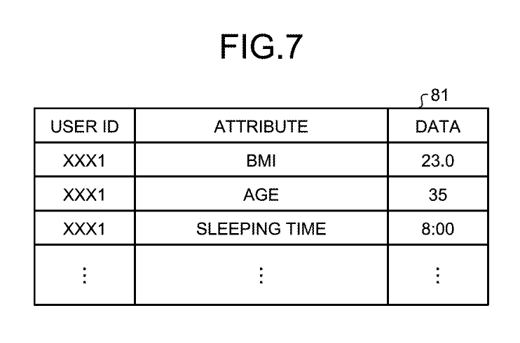

FIG. 7 is a diagram illustrating an example of a data structure of the user attribute information. The user attribute information 81 has items of user ID, an attribute, and data. The item of the user ID is a region storing therein the user ID. The item of the attribute is a region storing therein identification information indicating an attribute type. Although the attribute is expressed by a name of the attribute type for making the attribute type easy to be known in the embodiment, for example, an attribute code is stored as the identification information indicating the attribute type. The item of the data is an item storing therein data of the attribute. The data of the attribute is stored in the item of the data. When the attributes are, for example, the BMI, the age, and the sleeping time, respective values thereof are stored in the item of the data.

The example of FIG. 7 indicates that for the driver the user ID of which is “XXX1”, the BMI is 23.0, the age is 35, and the sleeping time is 8 hours.

The operation plan information 82 is data storing information related to a future operation plan of the service vehicle 12 for each user. The operation plan information 82 stores, for example, information on an upcoming operation plan of the vehicle for each user.

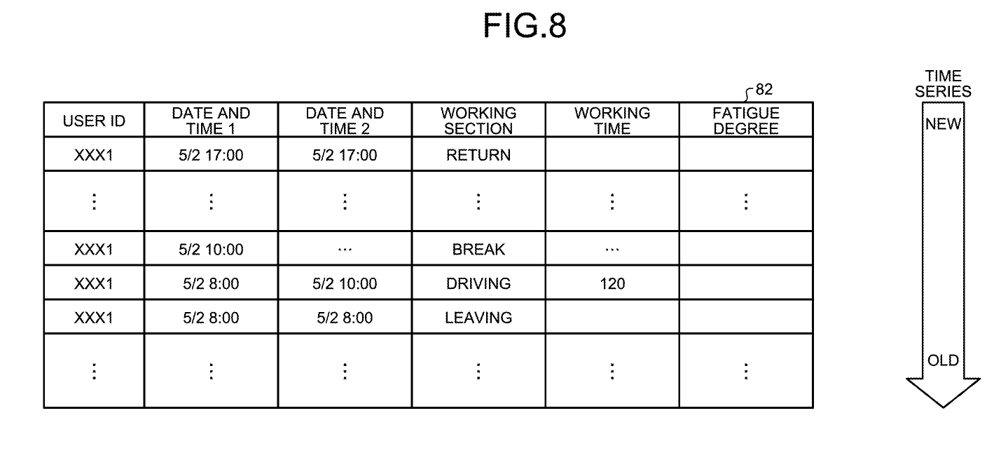

FIG. 8 is a diagram illustrating an example of a data structure of the operation plan information. The operation plan information 82 has a data structure that is similar to that of the work result information 80.

The example of FIG. 8 indicates that the driver the user ID of which is “XXX1” is planned to leave at 8:00 on May 2 in an upcoming operation plan of the vehicle. The example of FIG. 8 further indicates that the driver the user ID of which is “XXX1” is planned to drive from 8:00 to 10:00 on May 2 and the working time is 120 minutes.

The prediction model information 83 is data storing information related to a prediction model predicting the fatigue degree of the driver. Details of the prediction model will be described later.

The controller 72 controls the overall work plan support information provision apparatus 10. As the controller 72, an electronic circuit such as a CPU and an MPU or an integrated circuit such as an ASIC and an FPGA can be employed. The controller 72 has an internal memory for storing therein computer programs defining various processing procedures and pieces of control data and executes various pieces of processing by using them. The controller 72 functions as various processors by causing the various computer programs to be executed. The controller 72 includes, for example, a collector 90, a generator 91, a receiver 92, a calculator 93, and a presentation unit 94.

The collector 90 collects various pieces of data. The collector 90 collects, for example, the operation information 40 and the state information 41 from the operation recording meter 11. The collector 90 stores the collected operation information 40 and state information 41 in the storage unit 71.

The generator 91 generates the work result information 80 based on the state information 41. The generator 91, for example, reads records in the order of the date and time for each user from the state information 41 and specifies the selected operation states from the item of the data in order for the records in which the attribute is an operation status. Then, when any operation state is selected, the generator 91 treats the selected operation state as lasting in a period before a subsequent operation state is selected, specifies the start date and time and the end date and time of the operation state, and calculates a time length [min] from the start date and time to the end date and time. Furthermore, the generator 91 treats, as leaving, the timing of first selection of the operation state of driving after return is selected. As described above, the timing of detection of pressing of the button indicating “leaving” may be treated as leaving. The generator 91 treats, as return, the timing of selection of return. When leaving can be selected as the operation state, the timing of selection of leaving is treated as return. Then, the generator 91 stores the user ID of the user, the start date and time, the end date and time, the attribute indicating the operation state, and the time length [min] from the start date and time to the end date and time in the work result information 80 in a correspondence manner. When, for example, the state information 41 is as illustrated in FIG. 4, for the driver user ID of which is “XXX1”, selection of driving as the operation state at 8:00 on May 1 corresponds to the timing of first selection of the operation state of driving after “return” is selected. The generator 91 therefore stores a record of the user ID “XXX1”, the date and time 1 “8:00 on May 1”, the date and time 2 “8:00 on May 1”, and the working section “leaving” in the work result information 80. Furthermore, the generator 91 calculates a time length from “8:00 on May 1” as the start date and time of the operation state of driving to “10:00 on May 1” as the end date and time of the operation state of driving to be 120 [min] for the driver the user ID of which is “XXX1”. Then, the generator 91 stores a record of the user ID “XXX1”, the date and time 1 “8:00 on May 1”, the date and time 2 “10:00 on May 1”, the working section “driving”, and the working time “120 [min]” in the work result information 80.

The receiver 92 receives various operations. For example, when the receiver 92 receives access from the terminal apparatus 13, it transmits pieces of information on various operation screens to an access source, causes the terminal apparatus 13 as the access source to display the operation screens, and receives various operations through the operation screens. The receiver 92, for example, provides a Web site of operation management service. When the receiver 92 receives access to the Web site of the operation management service from the terminal apparatus 13, it transmits information on a log-in screen to the terminal apparatus 13 as the access source, causes the terminal apparatus 13 to display the log-in screen, and receives a log-in operation by input of a log-in ID and a password. When the receiver 92 receives the log-in operation, it compares the input log-in ID and password with a previously registered log-in ID and password for authentication. When the log-in ID and password are authenticated to be valid, the receiver 92 transmits information on a menu screen to the terminal apparatus 13 as the access source and causes the terminal apparatus 13 to display the menu screen. On the menu screen, for example, a plan registration menu for registering a future operation plan and a fatigue degree presentation menu presenting the fatigue degree of the driver are provided.

When the plan registration menu is selected on the menu screen, the receiver 92 transmits information on an operation plan registration screen for registering the operation plan to the terminal apparatus 13 as the access source, causes the terminal apparatus 13 to display the operation plan registration screen, and receives registration of the operation plan.

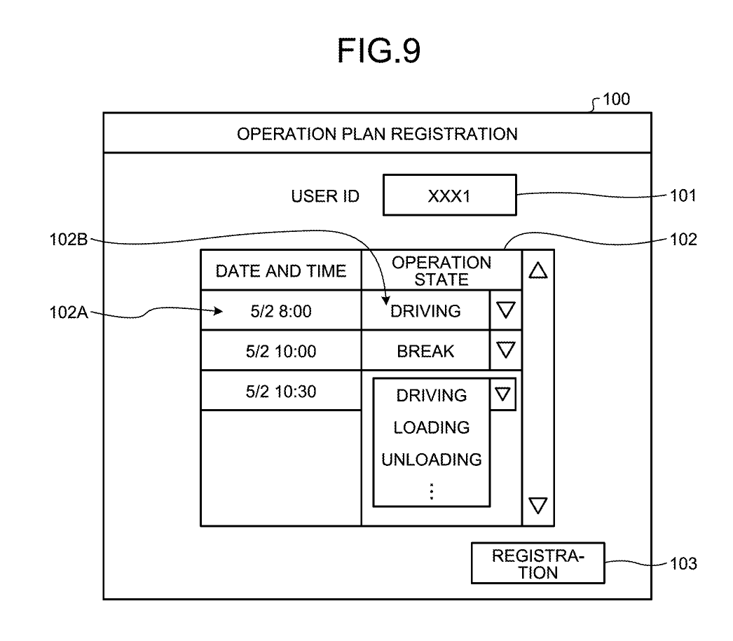

FIG. 9 is a diagram illustrating an example of the operation plan registration screen. The example of FIG. 9 illustrates an example of the operation plan registration screen that is displayed on the terminal apparatus 13. An operation plan registration screen 100 has an input region 101 for inputting the user ID of the user for which the operation plan is registered, an operation plan input region 102 for inputting the operation plan, and a registration button 103 for instructing to register the operation plan. The operation plan input region 102 has an input region 102A for inputting a date and time and a specification region 102B for specifying the operation state that is planned. The specification region 102B enables selection of the operation state from driving, loading, unloading, break, rest, and return by a selecting action. On the operation plan input region 102, a record in which the operation plan can be newly specified is displayed every specification of the operation plan for one record.

An operation manager inputs the user ID of the user for which the operation plan is registered to the input region 101 on the operation plan registration screen 100, inputs the future operation plan on the operation plan input region 102, and selects the registration button 103 using the terminal apparatus 13.

When the registration button 103 on the operation plan registration screen 100 is selected, the receiver 92 registers the operation plan input to the operation plan input region 102 in the operation plan information 82 so as to correspond to the user ID input to the input region 101. For example, the receiver 92 specifies the operation state, the start date and time, and the end date and time while, in the operation plan input region 102, the date and time in an upper record of two records aligned vertically in the input region 102A is the start date and time, the state specified in the specification region 102B for the upper record is the operation state, and the date and time in a lower record in the input region 102A is the end date and time. Then, the receiver 92 calculates the working time from the start date and time to the end date and time. The generator 91 stores the start date and time, the end date and time, the operation state, and the working time in the operation plan information 82 so as to correspond to the user ID input to the input region 101. The receiver 92 treats, as leaving, the timing of first selection of the operation state of driving and the receiver 92 treats, as return, the timing of selection of return. The receiver 92 stores the user ID of the user, the start date and time, the end date and time, the attribute indicating the operation state, and the time length [min] from the start date and time to the end date and time in the operation plan information 82 in a correspondence manner. When the operation plan registration screen 100 is, for example, as illustrated in FIG. 9, specification of the operation state of driving at 8:00 on May 2 is the timing of the first selection of the operation state of driving. The receiver 92 therefore stores a record of the user ID “XXX1”, the date and time 1 “8:00 on May 2”, the date and time 2 “8:00 on May 2”, and the working section “leaving” in the operation plan information 82. When the operation plan registration screen 100 is, for example, as illustrated in FIG. 9, the receiver 92 calculates the working time from the date and time to which the operation state of driving corresponds to the date and time to which the operation state of break corresponds. Then, the generator 91 stores a record of the user ID “XXX1”, the date and time 1 “8:00 on May 2”, the date and time 2 “10:00 on May 2”, the working section “driving”, and the working time “120 [min]” in the operation plan information 82.

When the fatigue degree presentation menu is selected on the menu screen, the receiver 92 transmits information on a user attribute registration screen for registering attribute information of the user to the terminal apparatus 13 as the access source, causes the terminal apparatus 13 to display the user attribute registration screen, and receives registration of the attribute information and the operation plan.

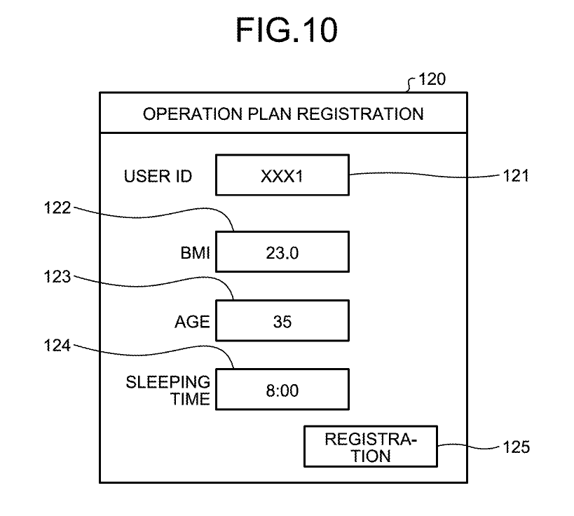

FIG. 10 is a diagram illustrating an example of the user attribute registration screen. The example of FIG. 10 illustrates an example of the user attribute registration screen that is displayed on the terminal apparatus 13. A user attribute registration screen 120 has an input region 121 for inputting the user ID of the user for which the attribute information of an individual is registered and various input regions for inputting various attributes that influence the fatigue. The user attribute registration screen 120 has, for example, an input region 122 for inputting the BMI, an input region 123 for inputting the age, an input region 124 for inputting the sleeping time, and the like. The user attribute registration screen 120 further has a registration button 125 for instructing to register the attribute information.

The operation manager inputs the user ID of the user for which the fatigue degree is presented to the input region 121 on the user attribute registration screen 120, inputs the pieces of attribute information of the user to the input regions 122 to 124, and selects the registration button 125 using the terminal apparatus 13.

When the registration button 125 on the user attribute registration screen 120 is selected, the receiver 92 registers the pieces of attribute information input to the input regions 122 to 124 in the user attribute information 81 so as to correspond to the user ID input to the input region 121.

The work plan support information provision apparatus 10 in the embodiment can estimate the fatigue degree in the case in which the driver performs transportation of an upcoming operation plan when the driver returns after transportation. For example, the operation manager refers to an estimation result of the fatigue degree in the case in which the driver performs the transportation of the upcoming operation plan using the work plan support information provision apparatus 10 in a return call when the driver returns after the transportation. Alternatively, the work plan support information provision apparatus 10 in the embodiment can estimate the fatigue degree in the case in which the driver performs transportation of an operation plan that has been the upcoming operation plan when the driver goes out for the transportation of the operation plan. For example, the operation manager refers to an estimation result of the fatigue degree in the case in which the driver performs the transportation of the operation plan using the work plan support information provision apparatus 10 in a leaving call when the driver departs for the transportation of the operation plan. Note that the sleeping time before the transportation of the upcoming operation plan is unknown in the return call. The input region 124 is therefore not input in the return call.

For the pieces of attribute information with less variations, pieces of master data may be stored in the storage unit 71 for each user without causing the user to input them every time. For example, the body height and the weight for deriving the BMI and the date of birth for deriving the age may be stored as pieces of master data in the storage unit 71 and read out from the pieces of master data because they vary little. Furthermore, the attribute information may be calculated from another piece of information if possible. Only the pieces of attribute information that are needed to be input may be input on the user attribute registration screen 120. For example, the input region 124 for inputting the sleeping time is not necessarily displayed on the user attribute registration screen 120 in the return call.

The calculator 93 performs various calculations. The calculator 93 calculates, for example, the fatigue degree of the user having the user ID input to the input region 121 on the user attribute registration screen 120 in an upcoming work time zone of the user using the prediction model stored in the prediction model information 83. In the embodiment, a prediction equation provided by multiple regression analysis or the like using, as explanatory variables, various pieces of information related to a past work result of the driver individual and various pieces of information related to the attribute information of the driver individual is stored in the prediction model information 83. As an example, an equation (1) indicates the prediction equation of the multiple regression analysis.

Y=b₁×X₁+b₂×X₂+b₃×X₃+ . . . +bₙXₙ+b₀ (1)

In this equation, Y is an objective variable. In the embodiment, the fatigue degree is the objective variable Y. X₁ to Xₙ are explanatory variables. In the embodiment, the various pieces of information related to the past work result and the various pieces of information related to the attribute information of the driver individual are the explanatory variables X₁ to Xₙ. b₁ to bₙ are coefficients. b₀ is a determinant. n is the number of pieces of information that are used as the explanatory variables.

In the embodiment, for example, the following explanatory variables are used.

Explanatory variable X₁: driving time

Explanatory variable X₂: break time

Explanatory variable X₃: rest time

Explanatory variable X₄: standby time

Explanatory variable X₅: loading time

Explanatory variable X₆: unloading time

Explanatory variable X₇: work interval time

Explanatory variable X₈: on-duty time

Explanatory variable X₉: previous fatigue degree

Explanatory variable X₁₀: BMI

Explanatory variable X₁₁: age

Explanatory variable X₁₂: sleeping time

The driving time, the loading time, the unloading time, and the on-duty time tend to increase the fatigue as they are longer. The break time, the rest time, and the standby time tend to decrease the fatigue as they are longer. The work interval time tends to increase the fatigue as it is shorter. The previous fatigue degree is a point of origin for calculation of the fatigue degree.

For example, the explanatory variables X₁ to Xₙ and the fatigue degree when a large number of drivers execute various operations are experimentally measured in advance. Values of b₀ to bₙ with which an error between Y provided from the equation (1) by performing the multiple regression analysis using the pieces of measured data and the fatigue degree is the minimum are calculated. The prediction model information 83 is stored in the storage unit 71 while information on the equation (1) to which the calculated values b₀ to bₙ are set is employed as the prediction model.

The calculator 93 calculates the explanatory variables X₁ to Xₙ by referring to the work result information 80 and the user attribute information 81.

The calculator 93 calculates, for example, a value by summing driving time lengths from the last leaving to return as the explanatory variable X₁.

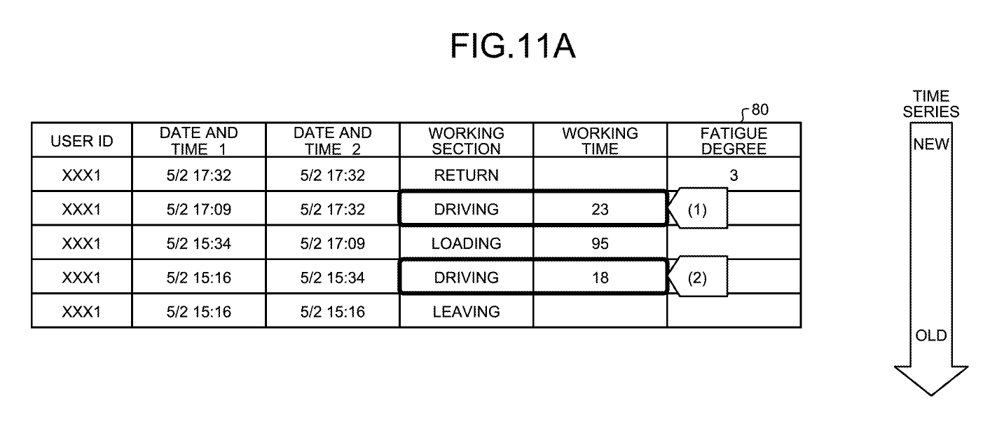

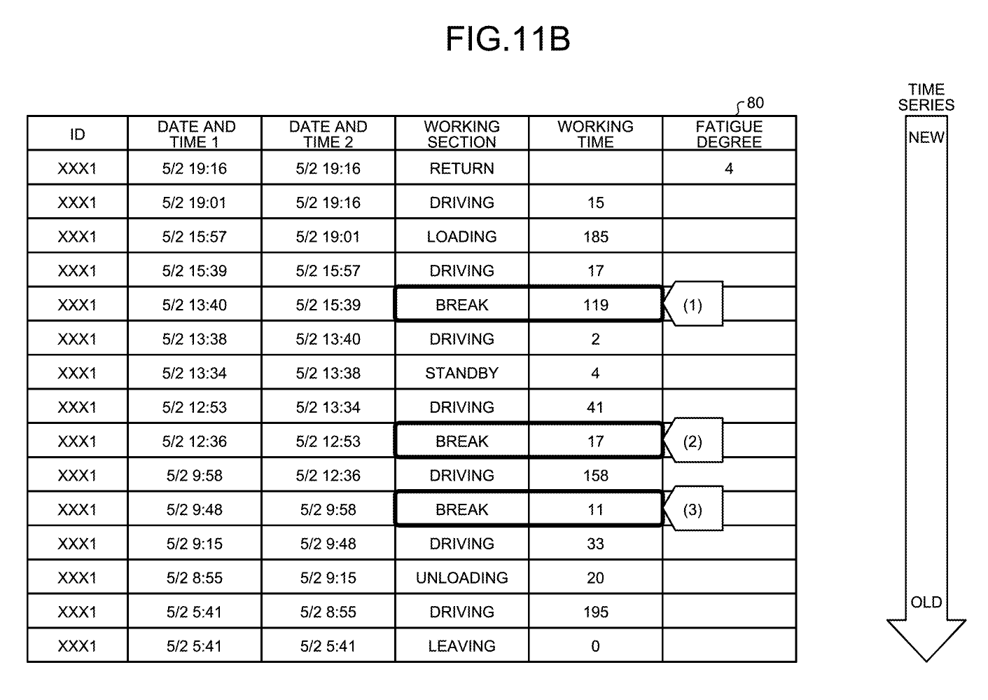

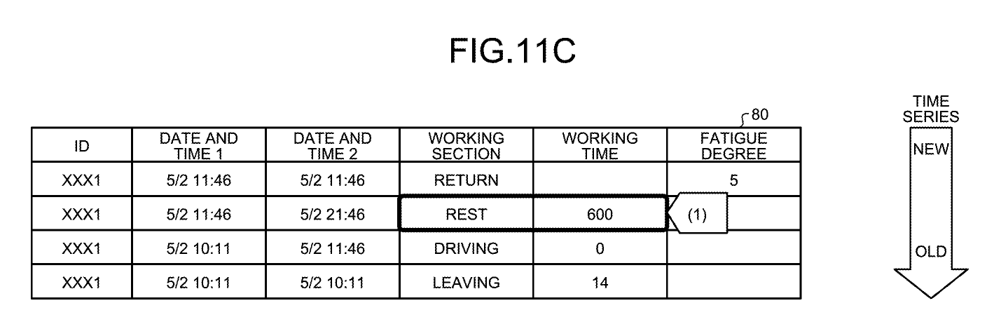

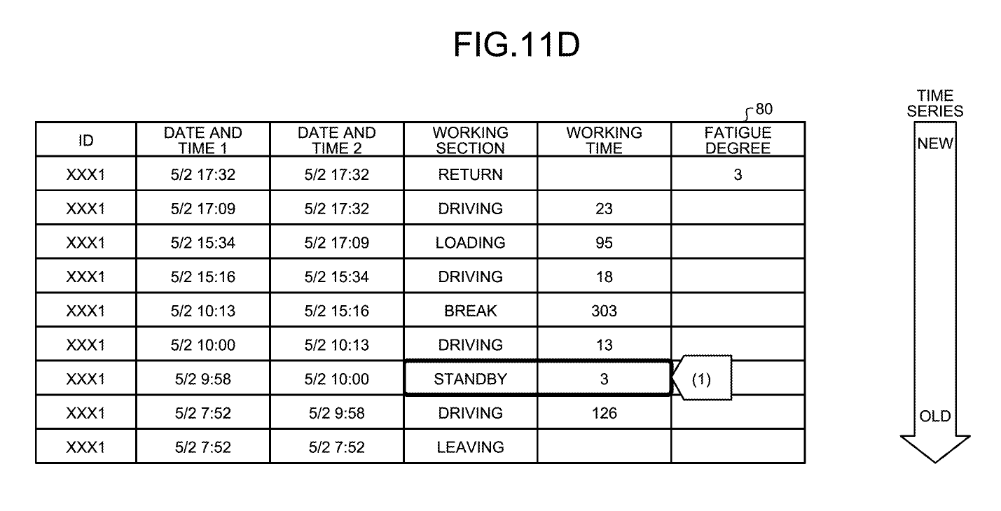

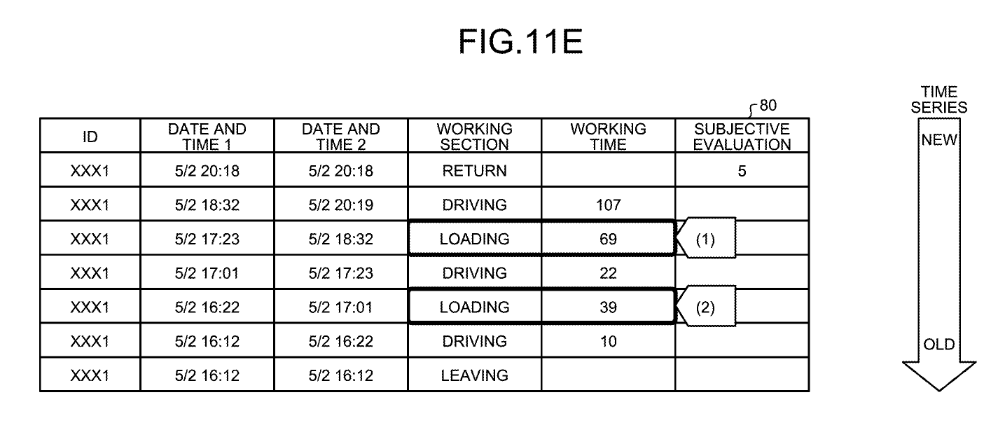

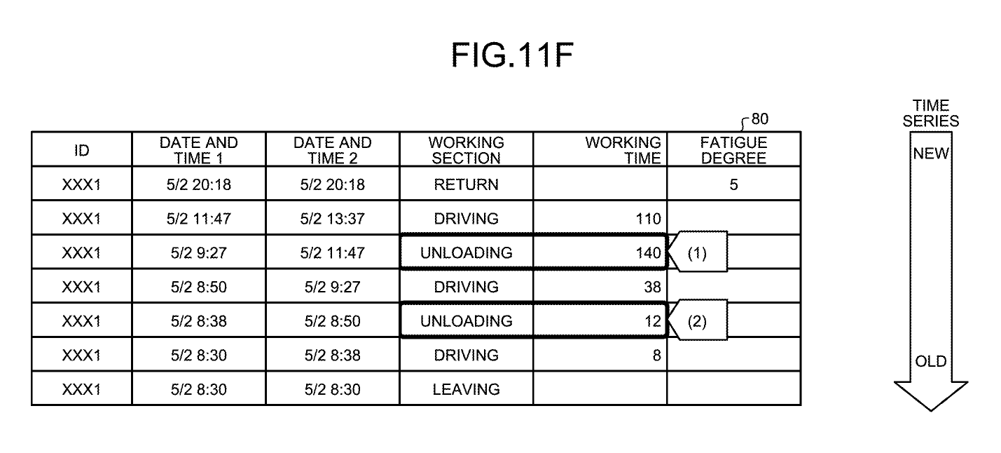

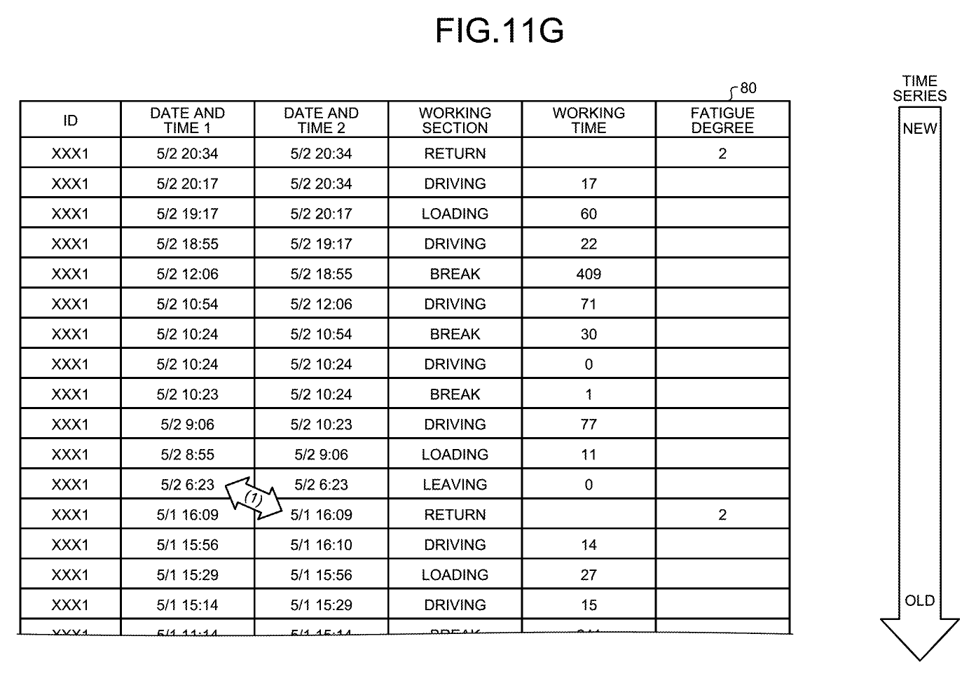

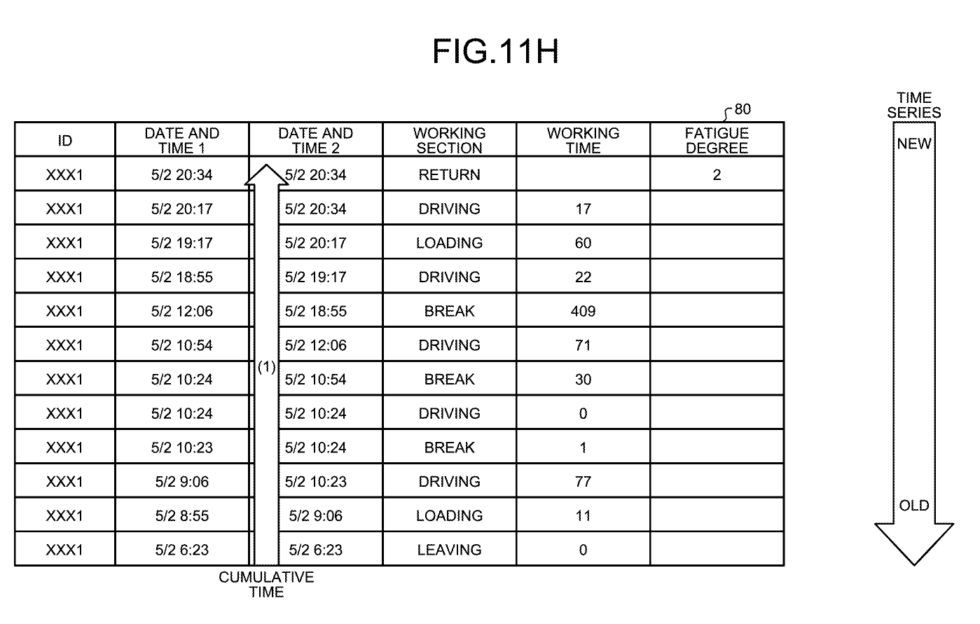

FIG. 11A is a diagram illustrating an example of calculation of driving time. FIG. 11A illustrates periods (1) and (2) in which the operation state is driving, as an example. The calculator 93 calculates a time length (min) by summing the periods (1) and (2) as the explanatory variable X₁.

The calculator 93 calculates a value by summing break time lengths from the last leaving to return as the explanatory variable X₂.

FIG. 11B is a diagram illustrating an example of calculation of the break time. FIG. 11B illustrates periods (1), (2), and (3) in which the operation state is break, as an example. The calculator 93 calculates a time length (min) by summing the periods (1), (2), and (3) as the explanatory variable X₂.

The calculator 93 calculates a value by summing rest time lengths from the last leaving to return as the explanatory variable X₃.

FIG. 11C is a diagram illustrating an example of calculation of the rest time. FIG. 11C illustrates a period (1) in which the operation state is rest, as an example. The calculator 93 calculates a time length (min) of the period (1) as the explanatory variable X₃.

The calculator 93 calculates a value by summing standby time lengths from the last leaving to return as the explanatory variable X₄.

FIG. 11D is a diagram illustrating an example of calculation of the standby time. FIG. 11D illustrates a period (1) in which the operation state is standby, as an example. The calculator 93 calculates a time length (min) of the period (1) as the explanatory variable X₄.

The calculator 93 calculates a value by summing loading time lengths from the last leaving to return as the explanatory variable X₅.

FIG. 11E is a diagram illustrating an example of calculation of the loading time. FIG. 11E illustrates periods (1) and (2) in which the operation state is loading, as an example. The calculator 93 calculates a time length (min) by summing the periods (1) and (2) as the explanatory variable X₅.

The calculator 93 calculates a value by summing unloading time lengths from the last leaving to return as the explanatory variable X₆.

FIG. 11F is a diagram illustrating an example of calculation of the unloading time. FIG. 11F illustrates periods (1) and (2) in which the operation state is unloading, as an example. The calculator 93 calculates a time length (min) by summing the periods (1) and (2) as the explanatory variable X₆.

The calculator 93 calculates a work interval time as the explanatory variable X₇. The calculator 93 calculates a time length (min) from the previous return to this leaving as the explanatory variable X₇.

FIG. 11G is a diagram illustrating an example of calculation of the work interval time. FIG. 11G illustrates a period (1) from the previous return to this leaving. The calculator 93 calculates a time length (min) of the period (1) as the explanatory variable X₇.

The calculator 93 calculates an on-duty time as the explanatory variable X₈.

FIG. 11H is a diagram illustrating an example of calculation of the on-duty time. FIG. 11H illustrates a period (1) from this leaving to this return. The calculator 93 calculates a time length (min) of the period (1) as the explanatory variable X₈.

The calculator 93 sets the previous fatigue degree as the explanatory variable X₉. The calculator 93 sets, for example, the fatigue degree in the previous return as the explanatory variable X₉. When the fatigue degree in the previous return is not registered, an initial value of the fatigue degree is input and the input initial value of the fatigue degree is set as the explanatory variable X₉.

The calculator 93 sets the explanatory variables X₁₀ to X₁₂ based on the user attribute information 81. Then, the calculator 93 calculates the fatigue degree of the driver from the equation (1) using the calculated explanatory variables X₁ to X₁₂.

The sleeping time before transportation of the upcoming operation plan is unknown in the return call. The calculator 93 sets the sleeping time by regarding a predetermined ratio (for example, 70%) of a period from the date and time of return to the date and time of leaving in the upcoming operation plan stored in the operation plan information 82 as a time length during which the driver sleeps. For example, when the date and time of return is 20:00 on May 1 and the date and time of leaving in the upcoming operation plan is 8:00 on May 2, the calculator 93 calculates a time length corresponding to a predetermined ratio of 12 hours from 20:00 on May 1 to 8:00 on May 2 and sets the calculated time length as the sleeping time. In this manner, the predicted fatigue degree of the driver at the time of leaving in the upcoming operation plan is calculated in the return call.

An actual sleeping time of the driver are set in the leaving call of the upcoming operation. By referring to this, the fatigue degree of the driver is calculated in the leaving call with higher accuracy than that in the return call.

Furthermore, the calculator 93 calculates fluctuations in the fatigue degree when the driver performs operation in accordance with the operation plan based on the operation plan information 82. The calculator 93 sets, for example, the predicted fatigue degree of the driver at the time of leaving as the explanatory variable X₉. The calculator 93 sets the explanatory variables X₁₀ to X₁₂ based on the user attribute information 81. The calculator 93 calculates the explanatory variables X₁ to X₈ at respective time points at which the operation state is switched in the operation plan of the operation plan information 82. The calculator 93 calculates the explanatory variables X₁ to X₈ at the respective time points of, for example, the dates and times in the item of the date and time 1 in the operation plan of the operation plan information 82. Then, the calculator 93 calculates the predicted fatigue degrees of the driver at the respective time points from the equation (1) using the calculated explanatory variables X₁ to X₈.

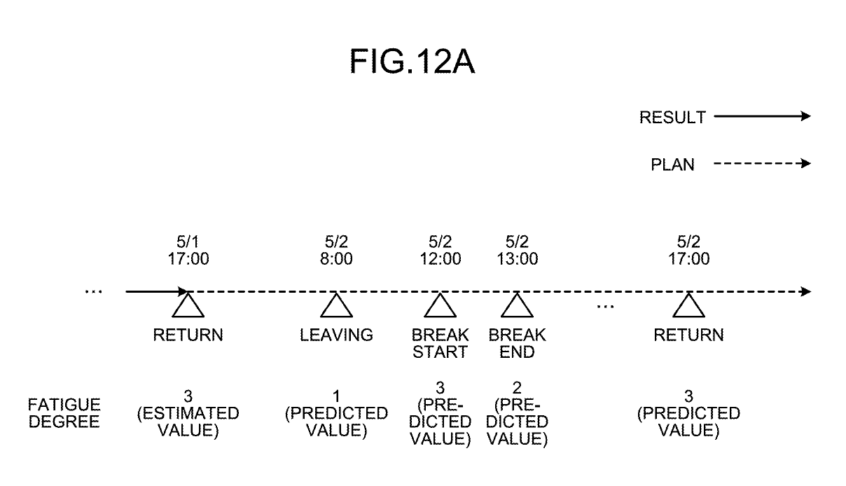

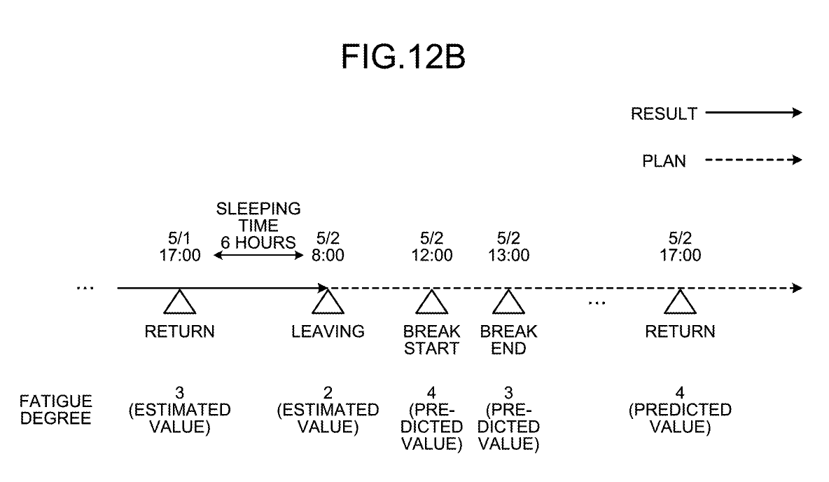

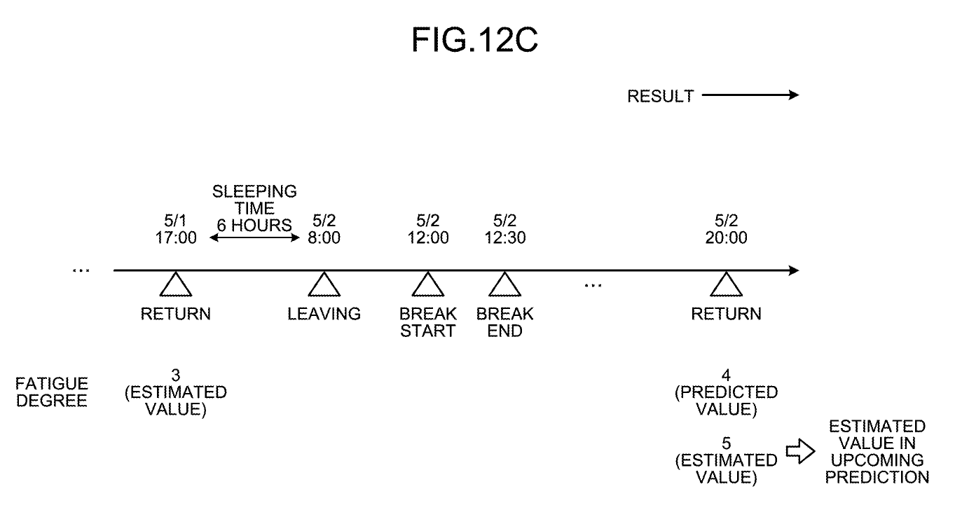

FIG. 12A is a diagram illustrating an example of calculation of the fatigue degree at the time of return. In the example of FIG. 12A, the fatigue degree of the driver at the time of return at 17:00 on May 1 is 3. The calculator 93 sets the explanatory variables X₁ to X₁₂ by referring to the work result information 80 and the user attribute information 81. In this case, the calculator 93 sets the sleeping time by regarding the predetermined ratio of a period from the date and time of return to the date and time of leaving in the upcoming operation plan stored in the operation plan information 82 as a time during which the driver sleeps. Then, the calculator 93 calculates the predicted fatigue degree of the driver at the time of leaving at 8:00 on May 2. In the example of FIG. 12A, the predicted fatigue degree of the driver at the time of leaving at 8:00 on May 2 is 1. The calculator 93 sets the predicted fatigue degree of the driver as the explanatory variable X₉ and calculates the predicted fatigue degrees of the driver at the respective time points at which the operation state is switched in the operation plan of the operation plan information 82. In the example of FIG. 12A, the fatigue degree of the driver at the start time of break at 12:00 on May 2 is calculated to be 3. Furthermore, the predicted fatigue degree of the driver at the end time of break at 13:00 on May 2 is calculated to be 2. The predicted fatigue degree of the driver at the time of return at 17:00 on May 2 is calculated to be 3.

FIG. 12B is a diagram illustrating an example of calculation of the fatigue degree at the time of leaving. In the example of FIG. 12B, the fatigue degree of the driver at the time of return at 17:00 on May 1 is 3. The calculator 93 sets the explanatory variables X₁ to X₁₂ by referring to the work result information 80 and the user attribute information 81. In this case, the calculator 93 sets an actual sleeping time. In the example of FIG. 12B, the actual sleeping time is 6 hours. The calculator 93 calculates the predicted fatigue degree of the driver at the time of leaving at 8:00 on May 2. In the example of FIG. 12B, the predicted fatigue degree of the driver at the time of leaving at 8:00 on May 2 is 2. This is because the sleeping time is as short as 6 hours and the driver does not sufficiently recover from fatigue. The calculator 93 sets the predicted fatigue degree of the driver as the explanatory variable X₉ and calculates the predicted fatigue degrees of the driver at the respective time points at which the operation state is switched in the operation plan of the operation plan information 82. In the example of FIG. 12B, the predicted fatigue degree of the driver at the start time of break at 12:00 on May 2 is calculated to be 4. Furthermore, the predicted fatigue degree of the driver at the end time of break at 13:00 on May 2 is calculated to be 3. The predicted fatigue degree of the driver at the time of return at 17:00 on May 2 is calculated to be 4.

The calculator 93 stores the calculated fatigue degrees at the respective time points in the item of the fatigue degree in the operation plan information 82. When the fatigue degree has been already stored, the calculator 93 overwrites it.

The operation plan stored in the operation plan information 82 and actual operation are different from each other in some cases. In this case, the fatigue degree of the driver that is predicted from the operation plan and the fatigue degree of the driver due to the actual operation are different from each other in some cases. In the example of FIG. 12B, when operation that is different from the operation plan is performed on May 2, the fatigue degree of the driver at the time of return on May 2 is different from the predicted fatigue degree in some cases. To cope with this, when the operation information 40 and the state information 41 for the actual operation relative to the operation plan are provided, the fatigue degree at the time of return is calculated again.

FIG. 12C is a diagram illustrating another example of calculation of the fatigue degree at the time of return. In the example of FIG. 12C, the actual operation does not proceed as scheduled in the operation plan illustrated in FIG. 12B due to traffic congestion or the like, and the break end comes earlier at 12:30 and the return time is 20:00. The calculator 93 sets the explanatory variables X₁ to X₁₂ related to the operation on May 2 by referring to the work result information 80 and the user attribute information 81. In this case, the fatigue degree at the time of return on May 1 is set as the explanatory variable X₉. Then, the calculator 93 calculates the fatigue degree of the driver at the time of return at 20:00 on May 2. The calculator 93 can thereby calculate the fatigue degree of the driver at the time of return on May 2 in accordance with the actual operation. In the example of FIG. 12B, the fatigue degree of the driver at the time of return on May 2 is predicted to be 4. On the other hand, in the example of FIG. 12C, as a result of recalculation in accordance with the actual operation, the fatigue degree of the driver at the time of return on May 2 is calculated to be 5.

The calculator 93 stores the fatigue degree provided by recalculation in accordance with the actual operation in the item of the fatigue degree in the work result information 80. In the example of FIG. 12C, the calculator 93 stores the fatigue degree provided by recalculation as the fatigue degree of the driver at the time of return on May 2 in the work result information 80. When the fatigue degree has been already stored, the calculator 93 overwrites it. The fatigue degree on May 3 is predicted using the fatigue degree of the driver at the time of return on May 2 as the previous fatigue degree (explanatory variable X₉).

As described above, the work plan support information provision apparatus 10 can prevent generation of an error in the fatigue degree by recalculating the fatigue degree in accordance with the actual operation.

The presentation unit 94 outputs various pieces of information. The presentation unit 94, for example, transmits information on a fatigue degree presentation screen containing the fatigue degrees calculated by the calculator 93 to the terminal apparatus 13 as the access source, and causes the terminal apparatus 13 to display the fatigue degree presentation screen.

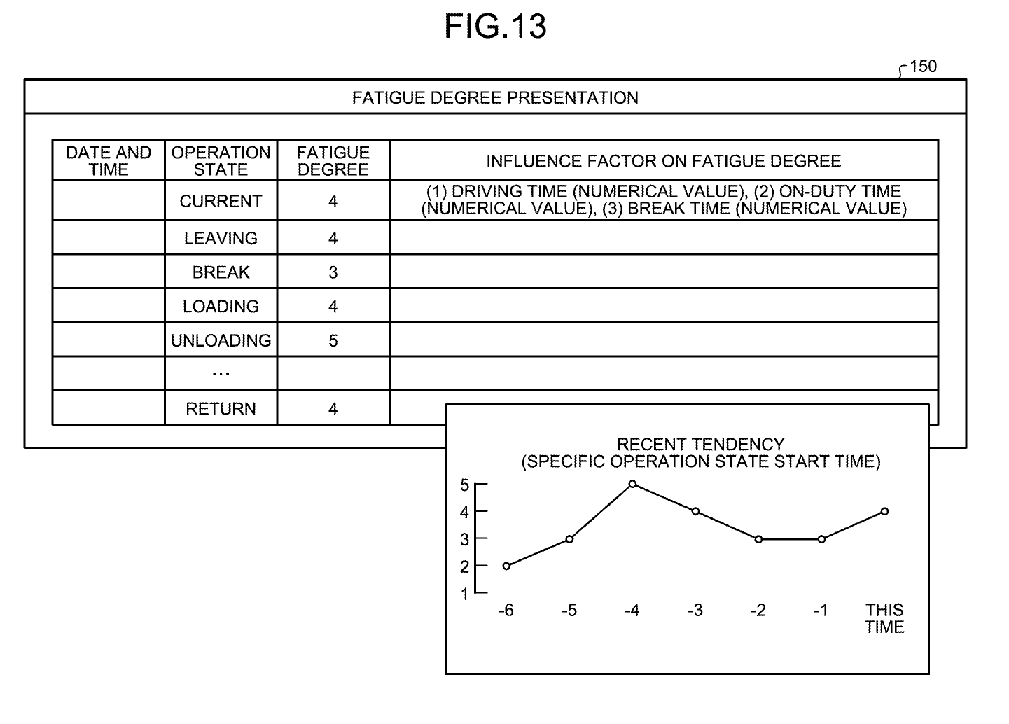

FIG. 13 is a diagram illustrating an example of the fatigue degree presentation screen. The example of FIG. 13 illustrates an example of the fatigue degree presentation screen that is displayed on the terminal apparatus 13. Transition of the fatigue degree along the operation plan is displayed on a fatigue degree presentation screen 150. In the embodiment, the degree of fatigue of the driver is expressed by the fatigue degrees of 1 to 5 and the fatigue degree of equal to or higher than a predetermined threshold (for example, 4) is regarded as a dangerous state for driving. The presentation unit 94 presents whether the fatigue degree is capable of being recovered with the fatigue degree. The fatigue degree of equal to or higher than 4 indicates a state incapable of recovering the fatigue degree and presenting inadequacy for work such as driving or necessity of caution. The fatigue degree of lower than the predetermined threshold indicates a state capable of recovering the fatigue degree and presenting capability of or adequacy for the work such as driving. The presentation unit 94 may present a notification indicating that driving is not recommended with a message or the like when the operation state for which the fatigue degree is equal to or higher than 4 is involved. The fatigue degree presentation screen 150 enables the operation manager to grasp the transition of the fatigue degree of the driver when the operation is performed in accordance with the operation plan, thereby grasping a dangerous operation with a high fatigue degree in advance and taking measures. The manager can determine whether the driver is to be in charge of operation of an upcoming operation plan by predicting and calculating the fatigue degrees of the driver in the upcoming operation in accordance with the upcoming operation plan in the return call of the driver, for example. Furthermore, when the fatigue degree of the driver is high, the manager can review the operation plan by delaying the leaving time and so on in order to prompt the driver to take a rest and recover from fatigue. For example, based on calculation of the fatigue degrees of the driver in accordance with the operation plan, the manager can advise, in the leaving call of the driver, the driver to take a break before the fatigue degree becomes high.

The presentation unit 94 outputs an element giving the largest influence on the calculated fatigue degree in the past work result of the driver individual and the attribute information of the driver individual. The presentation unit 94 outputs, for example, the attributes of the explanatory variables that derive values by multiplying the explanatory variables by the coefficients in the equation (1) that are equal to or higher than predetermined values, as the elements giving the largest influences on the calculated fatigue degrees and the fatigue degrees for the elements. The example of FIG. 13 presents, for the current fatigue degree, a driving time length, on-duty time length, and a rest time length and numerical values of the fatigue degrees for the respective elements as influence factors on the fatigue degree. The presentation unit 94 may present the predetermined number of upper order factors (for example, upper three factors) giving larger influences on the fatigue degrees.

The presentation unit 94 may present a history of the fatigue degrees calculated in the last predetermined period for a specific operation state. The example of FIG. 13 presents transition of the last six fatigue degrees as recent tendency for the specific operation state (for example, loading), as an example. The presentation enables fluctuations in the fatigue degree of the driver for the specific operation state to be grasped.

Processing Flow

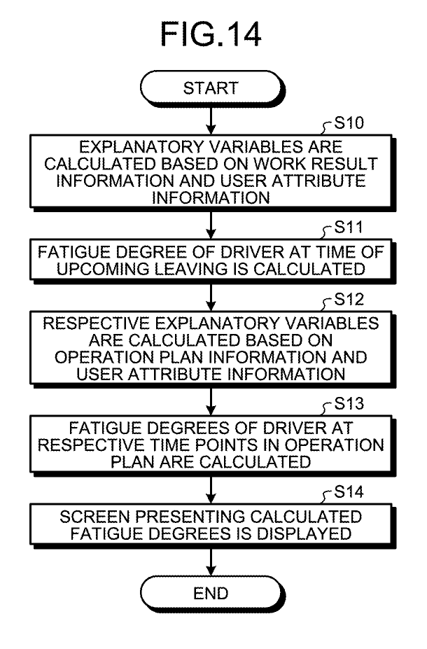

Next, flow of work plan support information provision processing that the work plan support information provision apparatus 10 in the embodiment executes will be described. FIG. 14 is a flowchart illustrating an example of procedures of the work plan support information provision processing. The work plan support information provision processing is executed at a predetermined timing. The work plan support information provision processing is executed at, for example, the timing when the user ID of the user for which the fatigue degrees are to be presented is input to the input region 121 on the user attribute registration screen 120, the pieces of attribute information are input to the input regions 122 to 124, and the registration button 125 is selected.

As illustrated in FIG. 14, the calculator 93 calculates the respective explanatory variables by referring to the work result information 80 and the user attribute information 81 (S10). The calculator 93 sets, for example, the fatigue degree of the driver at the time of return as the explanatory variable X₉ based on the work result information 80. The calculator 93 sets the explanatory variables X₁₀ to X₁₂ based on the user attribute information 81. Furthermore, the calculator 93 calculates the explanatory variables X₁ to X₈ based on the work result information 80.

The calculator 93 calculates the fatigue degree of the driver at the time of the upcoming leaving from the equation (1) using the respective calculated explanatory variables (S11).

The calculator 93 calculates the respective explanatory variables by referring to the operation plan information 82 and the user attribute information 81 (S12). The calculator 93 sets, for example, the predicted fatigue degree of the driver at the time of leaving as the explanatory variable X₉. The calculator 93 sets the explanatory variables X₁₀ to X₁₂ based on the user attribute information 81. Furthermore, the calculator 93 sets the explanatory variables X₁ to X₈ at the respective time points at which the operation state is switched in the operation plan of the operation plan information 82.

The calculator 93 calculates the predicted fatigue degrees of the driver at the respective time points in the operation plan from the equation (1) using the respective explanatory variables (S13).

The presentation unit 94 transmits information on the fatigue degree presentation screen presenting the calculated fatigue degrees to the terminal apparatus 13 as the access source and causes the terminal apparatus 13 to display the fatigue degree presentation screen 150 (S14). Then, the processing is finished.

Effects

As described above, the work plan support information provision apparatus 10 in the embodiment calculates the fatigue degrees of an individual for a plurality of types of operations in a future work time zone including the types of operations based on the past work result of the individual and the attribute information of the individual by referring to the storage unit 71 storing therein the past work result and the attribute information. The work plan support information provision apparatus 10 outputs the calculated fatigue degrees so as to correspond to the respective types of operations included in the future work time zone. The work plan support information provision apparatus 10 can thereby prevent occurrence of an accident.

Furthermore, the work plan support information provision apparatus 10 in the embodiment calculates fluctuations in the fatigue degree based on the past work result and the attribute information of the individual using the fatigue degree at the past predetermined timing as the initial value to calculate the fatigue degrees of the individual in the future work time zone. This calculation enables the work plan support information provision apparatus 10 to, for example, support determination by the operation management whether the upcoming work is adequate based on the calculated fatigue degrees in the upcoming work time zone, thereby preventing occurrence of an accident.

The work plan support information provision apparatus 10 in the embodiment calculates the predicted fatigue degrees of the individual when the vehicle is operated in accordance with the operation plan of the operation plan information of the vehicle for the individual that is stored in the storage unit 71 based on the future operation plan information 82. This calculation enables the work plan support information provision apparatus 10 to, for example, support determination by the operation management whether the operation plan is adequate based on the fatigue degrees of the individual that are predicted in accordance with the operation plan, thereby preventing occurrence of an accident.

The work plan support information provision apparatus 10 in the embodiment outputs the element giving the largest influence on the calculated fatigue degrees in the past work result of the individual and the attribute information of the individual. With this output, the work plan support information provision apparatus 10 can cause the operation manager or the driver to grasp a factor giving a large influence on the fatigue of the driver, thereby prompting improvement of the factor giving the large influence. The work plan support information provision apparatus 10 can thereby prevent occurrence of an accident.

The work plan support information provision apparatus 10 in the embodiment presents the history of the fatigue degrees calculated in the last predetermined period. This presentation enables the work plan support information provision apparatus 10 to provide tendency of the fluctuations in the fatigue, such as tendency of increase or decrease of the fatigue, based on the presented history of the fatigue degrees. The operation manager can therefore create the operation plan compatible with the future tendency of the fluctuations in the fatigue.

### [b] Second Embodiment

Although the embodiment related to the apparatus that is disclosed has been described hereinbefore, the disclosed technique may be implemented in various different modes other than the above-mentioned embodiment. The following describes other embodiments encompassed in the present invention.

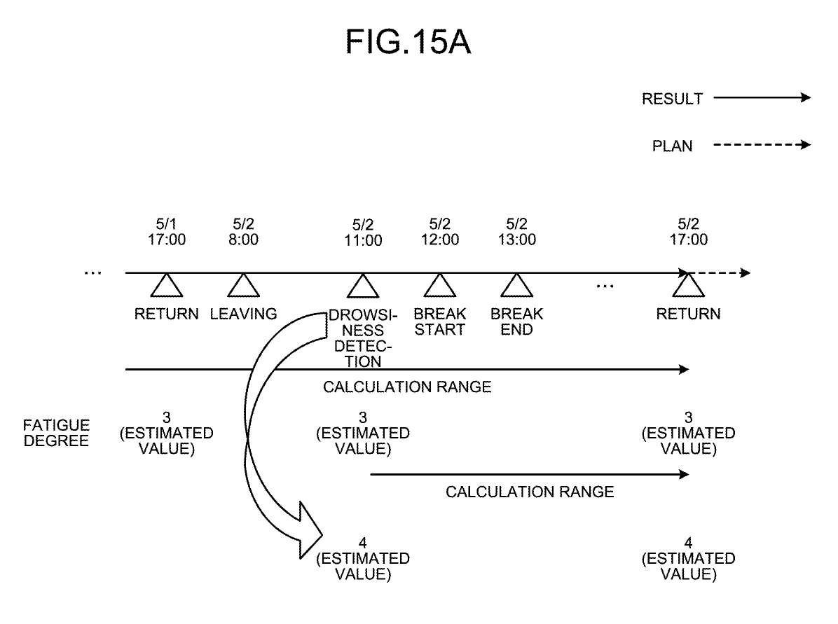

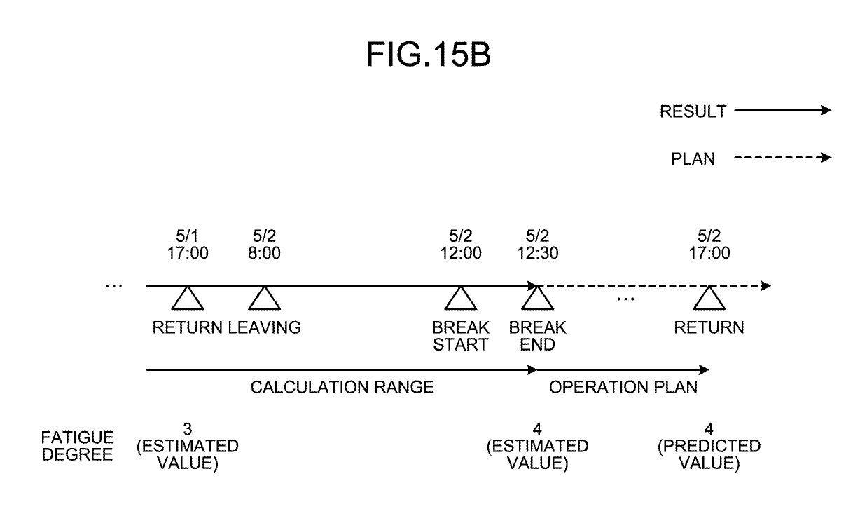

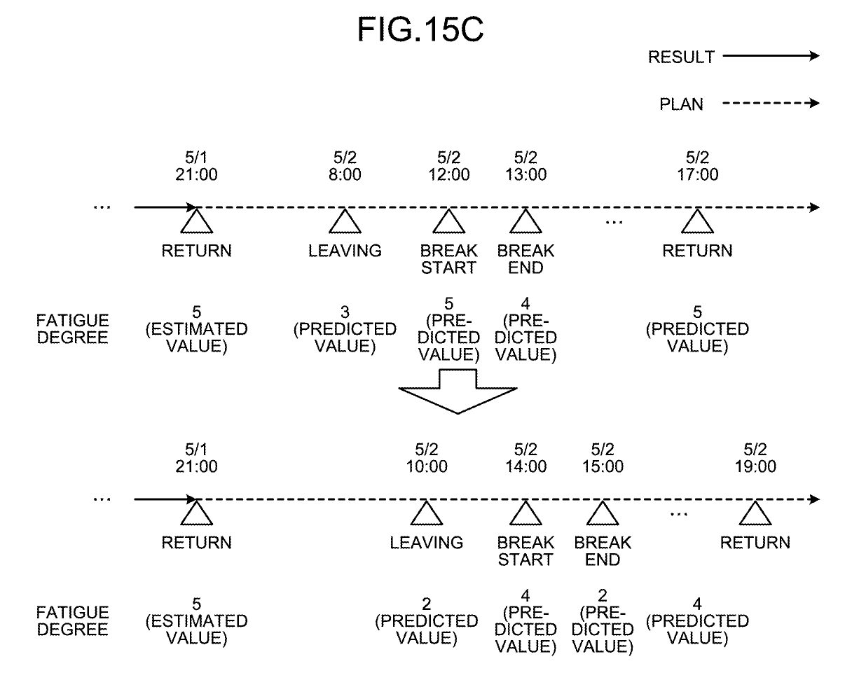

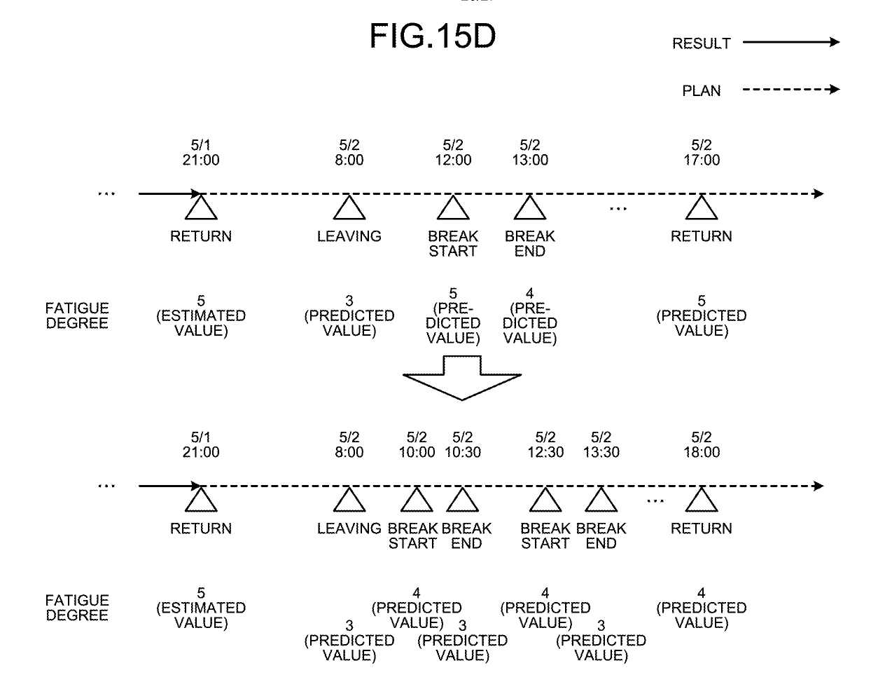

In the above-mentioned embodiment, for example, the fluctuations in the fatigue degree are calculated using the fatigue degree at the time of return in each operation as the initial value of the fatigue degree in the prediction equation after the initial value of the fatigue degree is set. The calculation of the fluctuations in the fatigue degree is however not limited thereto. For example, the calculator 93 may update the initial value of the fatigue degree of the individual based on the state information 41 indicating the state of the individual detected in the operation. Then, the calculator 93 may calculate the fatigue degrees of the individual based on the work result after the timing when the state of the individual is detected and the attribute information of the individual. For example, as the fatigue degree of the driver is higher, the driver's attentiveness tends to be diminished and deviation from a white line tends to occur. As the fatigue degree of the driver is higher, the drowsiness also tends to be generated. To cope with this, when information on detection of the deviation from a white line or the drowsiness is provided from the operation recording meter 11, the calculator 93 sets the fatigue degree at the detection timing of the deviation from a white line or the drowsiness to a value (for example, 4) corresponding to generation of the deviation from a white line or the drowsiness. The calculator 93 may calculate the fatigue degrees of the individual based on the work result after the detection timing of the deviation from a white line or the drowsiness and the attribute information of the individual. FIG. 15A is a diagram illustrating an example of calculation of the fatigue degrees when the drowsiness is detected. In the example of FIG. 15A, when the fatigue degree at the time of return on May 2 is calculated based on the work result after return on May 1 that has been stored in the work result information 80 using the fatigue degree 3 at the time of return on May 1 as the initial value of the fatigue degree, the fatigue degree is calculated to be 3. In the example of FIG. 15A, when the fatigue degree 3 at the time of return on May 1 is set to the initial value of the fatigue degree, the fatigue degree at 11:00 on May 2 is calculated to be 3. In the example of FIG. 15A, the drowsiness is detected at 11:00 on May 2. In this case, the calculator 93 updates the fatigue degree at 11:00 on May 2 to 4. Then, the calculator 93 calculates the fatigue degree at the time of return on May 2 based on the work result after 11:00 on May 2 that has been stored in the work result information 80 using the fatigue degree 4 at 11:00 on May 2 as the initial value of the fatigue degree. In the example of FIG. 15A, the fatigue degree at the time of return on May 2 is calculated to be 4. In this manner, the work plan support information provision apparatus 10 updates the initial value of the fatigue degree in accordance with detection of the deviation from a white line or the drowsiness, thereby making the calculated fatigue degree close to a fatigue state of the driver.

Furthermore, in the above-mentioned embodiment, the fatigue degrees are calculated in accordance with the operation plan at the time of leaving and return. The fatigue degrees are not however limited to be calculated in this manner. For example, the work plan support information provision apparatus 10 may collect the operation information 40 and the state information 41 from the operation recording meter 11 as appropriate and calculate the latest fatigue degree in real time based on the collected operation information 40 and state information 41. FIG. 15B is a diagram illustrating an example of calculation of the fatigue degree in real time. In the example of FIG. 15B, the operation information 40 and the state information 41 until 12:30 on May 2 are collected at 12:30 on May 2 and the work result is stored in the work result information 80. The calculator 93 calculates the fluctuations in the fatigue degree based on the work result until 12:30 on May 2 using the fatigue degree 3 at the time of return on May 1 as the initial value of the fatigue degree in the prediction equation. In the example of FIG. 15B, the fatigue degree at 12:30 on May 2 is 4. The calculator 93 calculates the fluctuations in the fatigue degree when the driver operates in accordance with the operation plan after 12:30 on May 2 based on the operation plan information 82 using the fatigue degree 4 at 12:30 on May 2 as the initial value of the fatigue degree in the prediction equation. This calculation enables the work plan support information provision apparatus 10 to provide the fatigue degrees to which the actual operation state is reflected in real time.

In the above-mentioned embodiment, the fatigue degrees are calculated in accordance with the operation plan. The fatigue degrees are not however limited to be calculated in this manner. For example, the operation plan may be automatically modified based on the fatigue degrees calculated in accordance with the operation plan. For example, the controller 72 of the work plan support information provision apparatus 10 may include a modification unit modifying the operation plan. The modification unit makes modification, on the operation plan, of delaying the operation plan overall when the fatigue degree calculated in accordance with the operation plan is equal to or higher than an acceptable level (for example, fatigue degree 5) or adding a break before the fatigue degree becomes equal to or higher than the acceptable level. FIG. 15C is a diagram illustrating an example of modification of the operation plan in accordance with the fatigue degree. In the example of FIG. 15C, the fatigue degree calculated in accordance with the operation plan is 5 at 12:00 on May 2. In this case, the modification unit delays the operation plan overall such that the calculated fatigue degree is lower than the predetermined acceptable level. In the example of FIG. 15C, the operation plan is changed to be delayed for two hours overall. FIG. 15D is a diagram illustrating another example of modification of the operation plan in accordance with the fatigue degree. Also in the example of FIG. 15D, the fatigue degree calculated in accordance with the operation plan is 5 at 12:00 on May 2. In this case, the modification unit makes modification, on the operation plan, of adding a break before the fatigue degree becomes 5. The break may be added immediately before a time point when the fatigue degree becomes 5. When there is a break or a rest after the time point at which the fatigue degree becomes 5, the break may be added at time point a constant period before the later break or rest. When there is a break or a rest before the time point at which the fatigue degree becomes 5, the break may be added at a time point a constant period after the previous break or rest or at an intermediate time point between the previous break or rest and the time point at which the fatigue degree becomes 5. In the example of FIG. 15D, a break is added at 10:00 to 10:30 on May 2.

In the above-mentioned embodiment, the fatigue degree is calculated using the common prediction model to all drivers. The fatigue degree is however not limited to be calculated in this manner. The fatigue degree may be calculated using, for example, individual prediction models for respective drivers. Furthermore, the prediction model may be updated. The work plan support information provision apparatus 10, for example, collects the fatigue degrees from the driver through the operation recording meter 11. The work plan support information provision apparatus 10 may update the prediction model based on the collected fatigue degrees of the driver and the calculated fatigue degrees. The calculator 93, for example, may update the coefficients b₁ to bₙ and the determinant b₀ in the equation (1) by performing the multiple regression analysis based on the collected fatigue degrees of the driver. In this case, the calculator 93 may update the individual prediction models for the respective drivers by performing the multiple regression analysis based on the collected fatigue degrees of the drivers. It is to be noted that the prediction model is not necessarily provided by the multiple regression analysis and may be constructed by various statistical methods, artificial intelligence (AI), deep learning, or the like.

In the above-mentioned embodiment, the work plan support information provision apparatus 10 collects pieces of information from the operation recording meter 11. The work plan support information provision apparatus 10 is however not limited to collect them in this manner. For example, exclusive application software may be installed on a mobile information terminal such as a smart phone and may be made to function as an operation recording meter.

In the above-mentioned embodiment, the attribute information of the driver individual is input on the screen. The acquisition manner thereof is however not limited thereto. For example, the attribute information of the driver individual may be acquired from a measurement device measuring the attribute information. For example, the BMI of the driver individual may be acquired from a weight scale or a pressure gauge provided in an office for operating the service vehicle 12. The sleeping time may be acquired from a sleep meter in driver's home.

In the above-mentioned embodiment, the work result is provided by processing the information on the operation state of the state information 41. The provision manner thereof is however not limited thereto. For example, the information on the operation state of the state information 41 may be used as the work result.

The respective components of the respective apparatuses illustrated in the drawings are functionally conceptual and are not necessarily requested to be configured physically as illustrated in the drawings. That is to say, specific states of separation and integration of the respective devices are not limited to those in the drawings and a part or all of them may be physically or functionally separated or integrated based on a desired unit in accordance with various loads and usage conditions. For example, the respective processors of the collector 90, the generator 91, the receiver 92, the calculator 93, and the presentation unit 94 may be appropriately integrated. Furthermore, processing by each processor may be appropriately separated into a plurality of pieces of processing by a plurality of processors. All or any part of the processing function that is executed by each processor can be implemented by a CPU or a computer program analyzed and executed by the CPU, or can be implemented as hardware by a wired logic.

Work Plan Support Information Provision Program

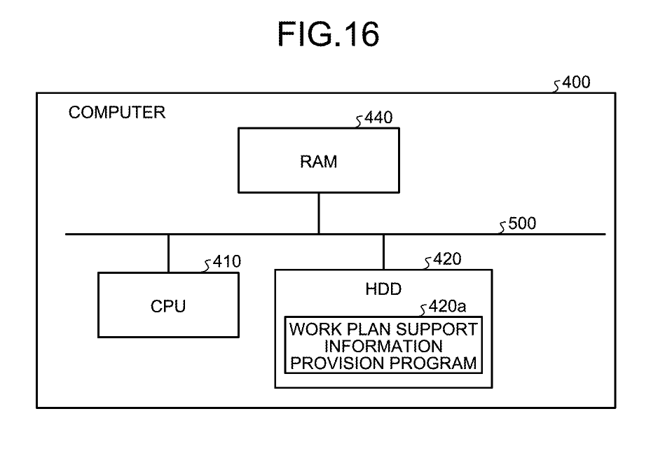

Various pieces of processing described in the above-mentioned embodiments can also be implemented by causing a computer system such as a personal computer and a work station to execute a previously prepared computer program. Hereinafter, an example of the computer system that executes the computer program having the same functions as those in the above-mentioned embodiments will be described. For example, a work plan support information provision program supporting creation of a work plan such as the operation plan of the service vehicle 12 will be described. FIG. 16 is a diagram illustrating an example of the configuration of a computer executing the work plan support information provision program.

As illustrated in FIG. 16, a computer 400 includes a central processing unit (CPU) 410, a hard disk drive (HDD) 420, and a random access memory (RAM) 440. The respective units 400 to 440 are connected to one another via a bus 500.

The HDD 420 previously stores therein a work plan support information provision program 420a executing the same functions as those of the respective processors (for example, the collector 90, the generator 91, the receiver 92, the calculator 93, and the presentation unit 94) of the above-mentioned controller 72. The work plan support information provision program 420a may be appropriately separated.

The HDD 420 stores therein various pieces of information. The HDD 420 stores therein, for example, an OS and various pieces of data that are used for determining an order quantity.

The CPU 410 executes the same operations as those of the respective processors in the embodiments by reading out the work plan support information provision program 420a from the HDD 420 and executing it. For example, the work plan support information provision program 420a executes the same operations as those of the collector 90, the generator 91, the receiver 92, the calculator 93, and the presentation unit 94.

The above-mentioned work plan support information provision program 420a is not necessarily requested to be stored in the HDD 420 from the beginning.

The work plan support information provision program 420a may be stored in a “portable physical medium” such as a flexible disk (FD), a compact disc read only memory (CD-ROM), a digital versatile disc (DVD), a magneto optical disk, and an integrated circuit (IC) card, that is inserted into the computer 400. The computer 400 may read the work plan support information provision program 420a from the above-mentioned medium and execute it.

Furthermore, the computer program is stored in “another computer (or a server)” that is connected to the computer 400 via a public network, the Internet, the local area network (LAN), the wide area network (WAN), or the like. The computer 400 may read the computer program from the above-mentioned computer and execute it.

Embodiments of the present invention provide an effect of outputting information indicating whether work to be allocated is adequate for a driver.

All examples and conditional language provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A work plan support information provision method comprising:
calculating fatigue degrees of an individual for a plurality of types of operations in a future work time zone including the types of operations based on a past work result of the individual and attribute information of the individual by referring to a storage storing therein the past work result and the attribute information, by a processor; and
outputting the calculated fatigue degrees so as to correspond to the respective types of operations included in the future work time zone, by the processor.

2. The work plan support information provision method according to claim 1, wherein the calculating includes calculating the fatigue degrees of the individual in the future work time zone by calculating fluctuations in the fatigue degree based on the past work result and the attribute information of the individual using the fatigue degree at a past predetermined timing as an initial value, by the processor.

3. The work plan support information provision method according to claim 1, wherein the calculating includes calculating predicted fatigue degrees of the individual when a vehicle is operated in accordance with an operation plan of future operation plan information of the vehicle for the individual that is stored in the storage, based on the operation plan information, by the processor.

4. The work plan support information provision method according to claim 2, further including updating the initial value of the fatigue degree of the individual based on state information indicating a state of the individual that has been detected in the operation of a vehicle, by the processor, wherein
the calculating includes calculating the fatigue degrees of the individual based on a work result after the timing when the state of the individual is detected and the attribute information of the individual, by the processor.

5. The work plan support information provision method according to claim 1, wherein the outputting includes outputting an element giving the largest influence on the calculated fatigue degrees in the past work result of the individual and the attribute information of the individual, by the processor.

6. The work plan support information provision method according to claim 1, wherein the outputting includes presenting a history of the fatigue degrees calculated in the last predetermined period, by the processor.

7. A non-transitory computer-readable recording medium storing therein a work plan support information provision program that causes a computer to execute a process comprising:
calculating fatigue degrees of an individual for a plurality of types of operations in a future work time zone including the types of operations based on a past work result of the individual and attribute information of the individual by referring to a storage storing therein the past work result and the attribute information; and
outputting the calculated fatigue degrees so as to correspond to the respective types of operations included in the future work time zone.

8. A work plan support information provision apparatus comprising:
a processor configured to:
calculate fatigue degrees of an individual for a plurality of types of operations in a future work time zone including the types of operations based on a past work result of the individual and attribute information of the individual by referring to a storage storing therein the past work result and the attribute information; and
output the fatigue degrees calculated so as to correspond to the respective types of operations included in the future work time zone.
